(19) United States
(12) Patent Application Publication
(10) Pub. No.: US 2025/0381313 A1
(43) Pub. Date: Dec. 18, 2025

(54) ULTRAVIOLET EMISSION UNIT

(71) Applicant: DAIKIN INDUSTRIES, LTD., Osaka (JP)

(72) Inventors: Chika KOYAMA, Osaka (JP); Tomoki SAITO, Osaka (JP); Tatsuki KOSHIYAMA, Osaka (JP); Ryuuji AKIYAMA, Osaka (JP)

(73) Assignee: DAIKIN INDUSTRIES, LTD., Osaka (JP)

(21) Appl. No.: 19/314,853

(22) Filed: Aug. 29, 2025

Related U.S. Application Data
(63) Continuation of application No. PCT/JP2024/008883, filed on Mar. 7, 2024.

(30) Foreign Application Priority Data
Mar. 8, 2023 (JP) 2023-035890

(51) Int. Cl. A61L 9/20; F24F 8/22

(52) U.S. Cl. CPC A61L 9/20; F24F 8/22; A61L 2209/12; A61L 2209/16

(57) ABSTRACT

An ultraviolet emission unit includes: an emission unit disposed on one end side in a first direction of the sterilization space and configured to emit an ultraviolet ray toward the other end side in the first direction; and a first reflection unit disposed on the other end side in the first direction of the sterilization space and configured to reflect the ultraviolet ray emitted from the emission unit such that the ultraviolet ray returns to the one end side in the first direction. An optical axis of light reflected by the first reflection unit is shifted from an optical axis of the ultraviolet ray of the emission unit by a first angle θ1 toward one end side in a second direction orthogonal to the first direction.

### TECHNICAL FIELD

The present disclosure relates to an ultraviolet emission unit, an air conditioner, and an air duct.

### BACKGROUND ART

An ultraviolet emission unit disclosed in Patent Document 1 includes an ultraviolet emitting diode that emits an ultraviolet ray, and a reflection unit that reflects the ultraviolet ray emitted from the ultraviolet emitting diode. As illustrated in FIG. 1 of this document, the ultraviolet ray emitted from the ultraviolet emitting diode is converted into parallel light and is then reflected by the reflection unit. The reflected ultraviolet light is sent to the ultraviolet emitting diode side along an optical axis of the parallel light. The air flowing between the ultraviolet emitting diode and the reflection unit is irradiated with the ultraviolet ray traveling back and forth between them, which inactivates bacteria and viruses in the air.

### CITATION LIST

### Patent Document

PATENT DOCUMENT 1: Japanese Unexamined Patent Publication No. 2022-160292

### SUMMARY

A first aspect is directed to an ultraviolet emission unit. The ultraviolet emission unit includes: a flow path forming member forming a sterilization space through which air flows; an emission unit disposed on one end side in a first direction of the sterilization space and configured to emit an ultraviolet ray toward the other end side in the first direction; and a first reflection unit disposed on the other end side in the first direction of the sterilization space and configured to reflect the ultraviolet ray emitted from the emission unit such that the ultraviolet ray returns to the one end side in the first direction. An optical axis of light reflected by the first reflection unit is shifted from an optical axis of the ultraviolet ray of the emission unit by a first angle θ1 toward one end side in a second direction orthogonal to the first direction.

### BRIEF DESCRIPTION OF THE DRAWINGS

FIG. 1 is a piping system diagram of an air conditioner according to an embodiment.

FIG. 2 is a perspective view illustrating an appearance of an indoor unit.

FIG. 3 is a cross-sectional view illustrating an internal structure of the indoor unit.

FIG. 4 is a perspective view illustrating a configuration of an ultraviolet emission unit.

FIG. 5 is a plan view illustrating an internal structure of the ultraviolet emission unit.

FIG. 6 is a schematic view of a configuration of an emission unit.

FIG. 7 is a schematic view of a configuration of an emission unit according to Variation 1A.

FIG. 8 is a schematic view of a configuration of an emission unit according to Variation 1B.

FIG. 9 is a plan view illustrating an internal structure of an ultraviolet emission unit according to Variation 2.

FIG. 10 is a plan view illustrating an internal structure of an ultraviolet emission unit according to Variation 3.

FIG. 11 is a perspective view illustrating a configuration of an ultraviolet emission unit according to Variation 4.

FIG. 12 is a plan view illustrating an internal structure of an ultraviolet emission unit according to Variation 5.

FIG. 13 is a plan view illustrating an internal structure of an ultraviolet emission unit according to Variation 6.

FIG. 14 is a plan view illustrating an internal structure of an ultraviolet emission unit according to an example of Variation 7.

FIG. 15 is a plan view illustrating an internal structure of an ultraviolet emission unit according to another example of Variation 7.

### DESCRIPTION OF EMBODIMENTS

Embodiments of the present disclosure will be described in detail below with reference to the drawings. The present disclosure is not limited to the embodiments shown below, and various changes can be made within the scope without departing from the technical concept of the present disclosure. Since each of the drawings is intended to illustrate the present disclosure conceptually, dimensions, ratios, or numbers may be exaggerated or simplified as necessary for the sake of ease of understanding.

### (1) Configuration of Air Conditioner

An ultraviolet emission unit (50) of the present disclosure is applied to an air conditioner (10). The air conditioner (10) conditions air in an indoor space which is a target space. The air conditioner (10) adjusts the temperature of the indoor air.

### (1-1) General Configuration

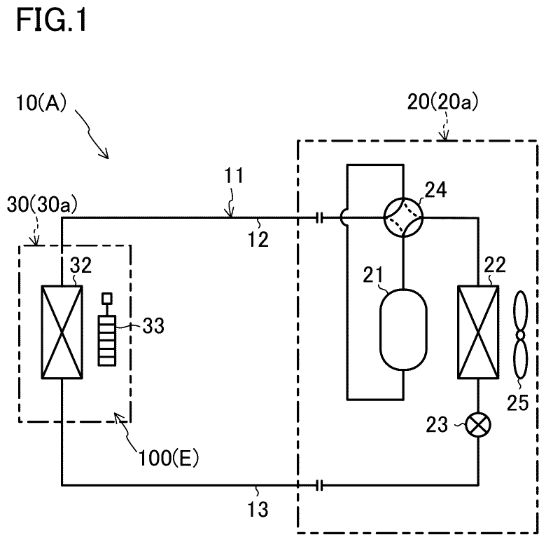

As illustrated in FIG. 1, the air conditioner (10) includes an outdoor unit (20), an indoor unit (30), a first connection pipe (12), and a second connection pipe (13). The air conditioner (10) is a pair-type air conditioner including one outdoor unit (20) and one indoor unit (30). The first connection pipe (12) is a gas connection pipe, and the second connection pipe (13) is a liquid connection pipe. The outdoor unit (20) and the indoor unit (30) are connected to each other via the first connection pipe (12) and the second connection pipe (13) to form a refrigerant circuit (11). The refrigerant circuit (11) circulates refrigerant therethrough to perform a refrigeration cycle. The refrigerant is, for example, difluoromethane.

### (1-2) Outdoor Unit

The outdoor unit (20) is installed outdoors. The outdoor unit (20) has an outdoor casing (20a), a compressor (21), an outdoor heat exchanger (22), an expansion valve (23), a four-way switching valve (24), and an outdoor fan (25). The outdoor casing (20a) houses the compressor (21), the outdoor heat exchanger (22), the expansion valve (23), the four-way switching valve (24), and the outdoor fan (25).

The compressor (21) is a rotary compressor of an oscillating piston type, a rotary type, a scroll type, or the like. The outdoor heat exchanger (22) is a fin-and-tube heat exchanger. The four-way switching valve (24) switches between a first state (state indicated by a solid line in FIG. 1) and a second state (state indicated by a broken line in FIG. 1). The four-way switching valve (24) in the first state causes a discharge portion of the compressor (21) and a gas end portion of the outdoor heat exchanger (22) to communicate with each other, and causes a suction portion of the compressor (21) and the first connection pipe (12) to communicate with each other. The four-way switching valve (24) in the second state causes the discharge portion of the compressor (21) and the first connection pipe (12) to communicate with each other, and causes the suction portion of the compressor (21) and the gas end portion of the outdoor heat exchanger (22) to communicate with each other. The outdoor fan (25) is a propeller fan.

### (1-3) Indoor Unit

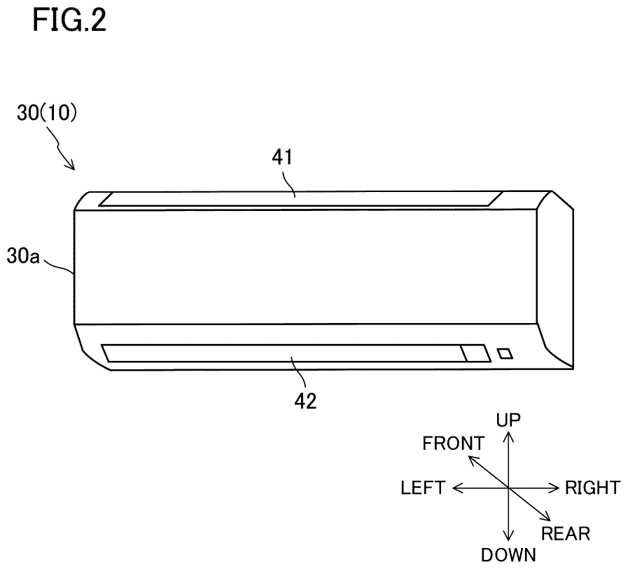

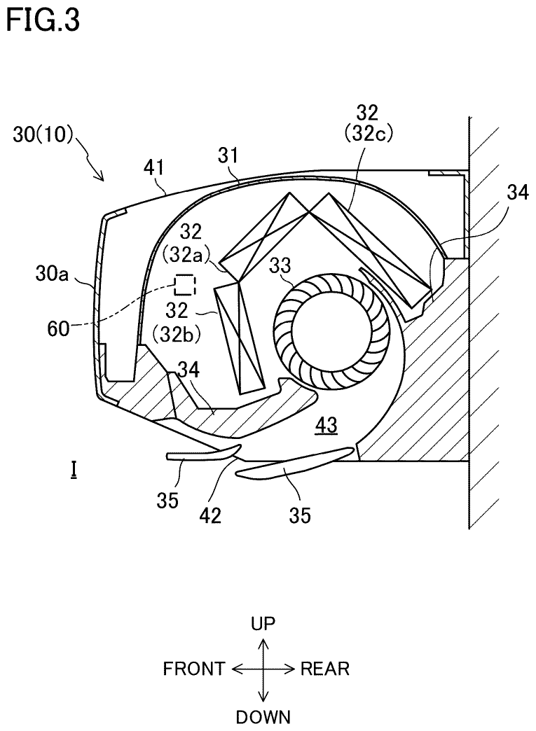

As illustrated in FIGS. 2 and 3, the indoor unit (30) is installed in the indoor space (I). The indoor unit (30) is a wall-mounted indoor air conditioner installed on a wall of the indoor space (I). The indoor unit (30) has an indoor casing (30a), an air filter (31), an indoor heat exchanger (32), an indoor fan (33), a drain pan (34), and a flap (35).

The indoor casing (30a) forms an air conditioning casing. The indoor casing (30a) is formed in a hollow shape that is long in the left-right direction. The longitudinal direction of the indoor casing (30a) is the left-right direction. The indoor casing (30a) houses the air filter (31), the indoor heat exchanger (32), the indoor fan (33), the drain pan (34), and the flap (35). The indoor casing (30a) has an inlet port (41) and an outlet port (42). The inlet port (41) is formed in an upper portion of the indoor casing (30a). The inlet port (41) is an opening through which air in the indoor space (I) is sucked. The inlet port (41) extends in the longitudinal direction of the indoor casing (30a). The outlet port (42) is formed near the front side in a lower portion of the indoor casing (30a). The outlet port (42) extends in the longitudinal direction of the indoor casing (30a). The indoor casing (30a) includes therein an air passage (43) from the inlet port (41) to the outlet port (42).

The air filter (31) is disposed upstream of the indoor heat exchanger (32) in the air passage (43). The air filter (31) is a mesh member formed along the inlet port (41). The air filter (31) collects dust in the air sucked through the inlet port (41).

The indoor heat exchanger (32) is disposed upstream of the indoor fan (33) in the air passage (43). The indoor heat exchanger (32) is a fin-and-tube heat exchanger. The indoor heat exchanger (32) allows heat exchange between the refrigerant flowing through the indoor heat exchanger (32) and air transferred by the indoor fan (33).

The indoor fan (33) is an example of a fan. The indoor fan (33) is a cross-flow fan. The indoor fan (33) extends in the longitudinal direction of the indoor casing (30a). The indoor fan (33) is rotationally driven by a fan motor (33a). The indoor fan (33) transfers the air in the air passage (43). When the indoor fan (33) is driven, air in the indoor space (I) is sucked into the air passage (43) and flows through the air passage (43). At the same time, the air in the air passage (43) is blown out through the outlet port (42). The indoor fan (33) is configured to be capable of adjusting the volume of blown air to be supplied to the indoor space (I) through the outlet port (42).

The drain pan (34) is disposed on the lower side of the indoor heat exchanger (32). The drain pan (34) is a tray which receives water generated in the indoor casing (30a). The drain pan (34) receives condensation water generated on the surface of the indoor heat exchanger (32).

The flap (35) forms a wind direction adjuster that adjusts the direction of the blown air. The flap (35) adjusts the direction of the blown air in the up-down direction. The flap (35) may adjust the direction of the blown air in the left-right direction.

### (2) Ultraviolet Emission Unit

The indoor unit (30) of the air conditioner (10) includes an ultraviolet emission unit (50). The ultraviolet emission unit (50) inactivates bacteria and viruses in air with ultraviolet light. As illustrated in FIG. 3, the ultraviolet emission unit (50) is housed in the indoor casing (30a). The ultraviolet emission unit (50) is disposed in the air passage (43). Specifically, the ultraviolet emission unit (50) is disposed upstream of the indoor heat exchanger (32) in the air passage (43).

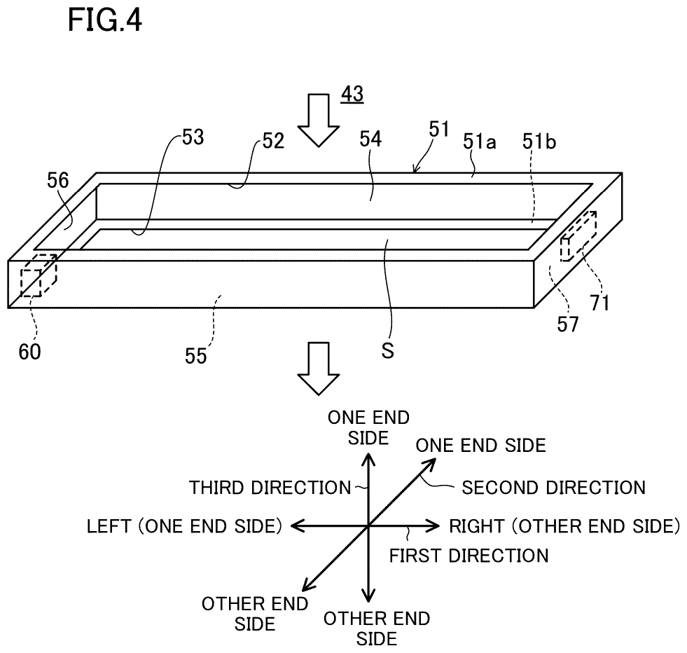

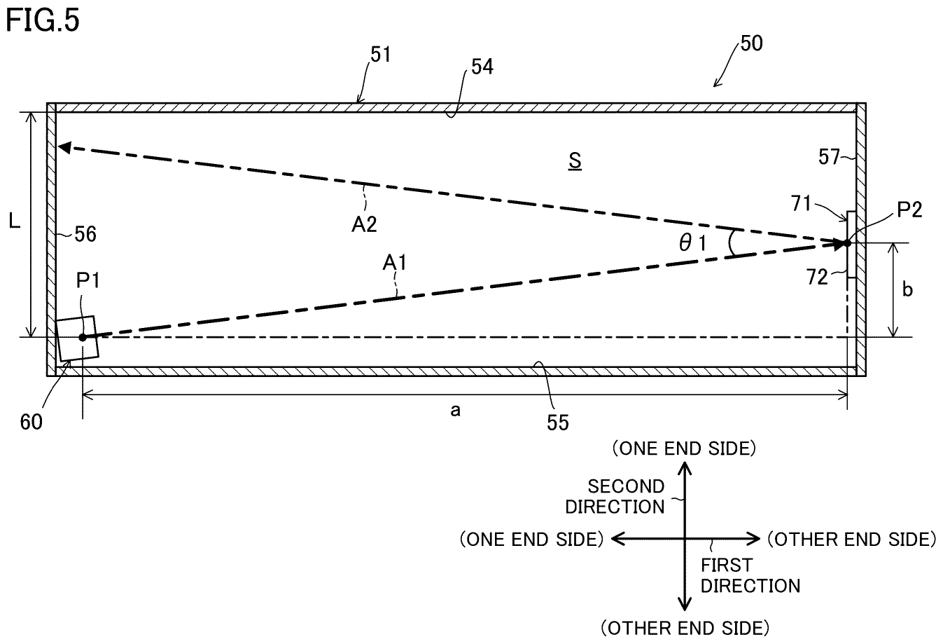

As illustrated in FIGS. 4 and 5, the ultraviolet emission unit (50) includes a casing (51) as a flow path forming member, an emission unit (60), and a first reflection unit (71).

### (2-1) Casing

A sterilization space (S) through which air to be sterilized with an ultraviolet ray flows is formed inside the casing (51). The casing (51) is formed in a hollow rectangular parallelepiped shape. The casing (51) extends along the longitudinal direction (left-right direction) of the indoor unit (30). As illustrated in FIG. 4, the longitudinal direction of the casing (51) corresponds to the left-right direction or longitudinal direction of the indoor casing (30a). In this embodiment, the thickness direction of the casing (51) corresponds to the direction of an air flow through the sterilization space (S). The width direction of the casing (51) corresponds to a direction orthogonal to the longitudinal direction and thickness direction of the casing (51).

An inflow port (52) is formed in a first casing surface (51a) on one end side in the thickness direction of the casing (51). The inflow port (52) is formed in the substantially entire area of the first casing surface (51a). An outflow port (53) is formed in a second casing surface (51b) on the other end side in the thickness direction of the casing (51). The outflow port (53) is formed in the substantially entire area of the second casing surface (51b). The sterilization space (S) is formed in the casing (51) from the inflow port (52) to the outflow port (53). The inflow port (52) and the outflow port (53) are opposed to each other through the sterilization space (S).

The inflow port (52) is opposed to the inlet port (41) of the indoor casing (30a). The inflow port (52) is opposed to the downstream surface of the air filter (31). The outflow port (53) is opposed to the air inflow surface of the indoor heat exchanger (32).

The sterilization space (S) is a rectangular parallelepiped space. The longitudinal direction of the sterilization space (S) corresponds to the left-right direction or longitudinal direction of the indoor casing (30a). The thickness direction of the sterilization space (S) corresponds to the direction of the air flow through the sterilization space (S). The width direction of the sterilization space (S) corresponds to a direction orthogonal to the longitudinal direction and thickness direction of the sterilization space (S). In this embodiment, the longitudinal direction of the sterilization space (S) is a first direction; the width direction of the sterilization space (S) is a second direction; and the thickness direction of the sterilization space (S) is a third direction.

Six inner surfaces facing the sterilization space (S) are formed inside the casing (51). As illustrated in FIGS. 4 and 5, the six inner surfaces include a first surface (54), a second surface (55), a third surface (56), and a fourth surface (57). The first surface (54) is formed on one end side of the sterilization space (S) in the second direction. The second surface (55) is formed on the other end side of the sterilization space (S) in the second direction. The third surface (56) is formed on one end side of the sterilization space (S) in the first direction. The fourth surface (57) is formed on the other end side of the sterilization space (S) in the first direction.

### (2-2) Emission Unit

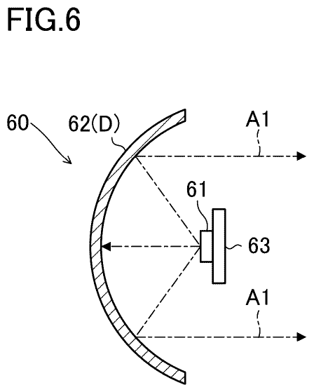

As illustrated in FIG. 6, the emission unit (60) includes a light emitting diode (LED) (61), a reflector (62) which is an example of a light distribution control unit (D), and a circuit board (63) that controls the LED (61).

The LED (61) is a light emitting source that emits the ultraviolet ray. The peak wavelength of the ultraviolet ray emitted from the LED (61) is 280 nm or less. This can enhance the sterilization effect on air. The peak wavelength of the ultraviolet ray emitted from the LED (61) is preferably 255 nm or more and 275 nm or less. This can particularly enhance the sterilization effect on air. The peak wavelength of the ultraviolet ray emitted from the LED (61) may be 230 nm or less. This can improve safety for the human body in terms of exposure in the event of ultraviolet ray leakage from the indoor casing (30a).

The reflector (62) is a curved reflection plate that reflects the ultraviolet ray emitted from the LED (61). In this embodiment, the LED (61) faces the reflector (62) side. The reflector (62) reflects the ultraviolet ray emitted from the LED (61), thereby distributing the ultraviolet ray emitted from the emission unit (60) such that the ultraviolet ray is directed toward a first optical axis (A1).

The circuit board (63) includes a control board for controlling the LED (61).

Specifically, the circuit board (63) includes a control device for switching the LED (61) between ON and OFF and adjusting the output of the LED (61). The control device of the circuit board (63) may be provided in an air conditioning controller for controlling the air conditioner (10).

The LED (61) and the circuit board (63) may be provided with a heat dissipation member for suppressing an increase in the temperature of the LED (61).

### (2-3) First Reflection Unit

The first reflection unit (71) reflects the ultraviolet ray emitted from the emission unit (60). Precisely, the first reflection unit (71) reflects the ultraviolet ray distributed by the light distribution control unit (D). The first reflection unit (71) is a reflection member having a reflection surface (72) facing the emission unit (60). The ultraviolet ray reflectance of the first reflection unit (71) is preferably 50% or more. Here, the reflectance R is expressed by Expression (1) below.

$$R [ \% ] = ( E 2 / E 1 ) \times 100 \qquad ( 1 )$$

Here, E1 is the amount of ultraviolet light [mW] entering the reflection unit, and E2 is the amount of ultraviolet light [mW] reflected by the reflection unit.

### (3) Operation of Air Conditioner

The air conditioner (10) performs a cooling operation and a heating operation.

### (3-1) Cooling Operation

The cooling operation is an operation for cooling air in the indoor space (I) so that the air in the indoor space (I) approaches a set temperature (target temperature). In the cooling operation, the four-way switching valve (24) is brought into the first state. The refrigerant compressed in the compressor (21) dissipates heat in the outdoor heat exchanger (22), and is then decompressed by the expansion valve (23). The decompressed refrigerant evaporates in the indoor heat exchanger (32). The air cooled in the indoor heat exchanger (32) is supplied to the indoor space (I). The refrigerant evaporated in the indoor heat exchanger (32) is sucked into the compressor (21).

### (3-2) Heating Operation

The heating operation is an operation for heating air in the indoor space (I) so that the air in the indoor space (I) approaches the set temperature (target temperature). In the heating operation, the four-way switching valve (24) is brought into the second state. In the heating operation, the refrigerant compressed in the compressor (21) dissipates heat in the indoor heat exchanger (32), and is then decompressed by the expansion valve (23). The air heated in the indoor heat exchanger (32) is supplied to the indoor space (I). The decompressed refrigerant evaporates in the outdoor heat exchanger (22), and is then sucked into the compressor (21).

### (4) Details of Layout of Ultraviolet Emission Unit

The layout of the emission unit (60) and the first reflection unit (71) in the sterilization space (S) will be described in detail with reference to FIGS. 4 and 5.

### (4-1) Emission Unit

The emission unit (60) is disposed on one end side in the first direction of the sterilization space (S). The emission unit (60) is disposed between an intermediate position in the first direction of the sterilization space (S) and the third surface (56). Specifically, the emission unit (60) is disposed in the vicinity of the third surface (56). The emission unit (60) is preferably fixed to the third surface (56).

The emission unit (60) is disposed near the second surface (55). The emission unit (60) is disposed between an intermediate position in the second direction of the sterilization space (S) and the second surface (55). The emission unit (60) is disposed in the vicinity of the second surface (55).

The emission unit (60) emits the ultraviolet ray from one end side to the other end side in the first direction of the sterilization space (S). The first optical axis (A1) of the ultraviolet ray of the emission unit (60) is directed to the fourth surface (57). The first optical axis (A1) is inclined to the first surface (54) side with respect to the first direction.

### (4-2) First Reflection Unit

The first reflection unit (71) is disposed on the other end side in the first direction of the sterilization space (S). The first reflection unit (71) is disposed between the intermediate position in the first direction of the sterilization space (S) and the fourth surface (57). The first reflection unit (71) is disposed in the vicinity of the fourth surface (57). The first reflection unit (71) is disposed at the intermediate position in the second direction of the sterilization space (S). The first reflection unit (71) is preferably fixed to the fourth surface (57).

The first reflection unit (71) reflects the ultraviolet ray toward the one end side in the first direction of the sterilization space (S) from the other end side. The first reflection unit (71) has a reflection surface (71a) facing one end side in the first direction and reflecting the ultraviolet ray. A second optical axis (A2) of the ultraviolet ray reflected by the first reflection unit (71) is directed to the third surface (56). The second optical axis (A2) is inclined to the first surface (54) side with respect to the first direction. The ultraviolet ray reflected by the first reflection unit (71) returns to the one end side in the first direction of the sterilization space (S). The ultraviolet ray reflected by the first reflection unit (71) reaches the third surface (56).

### (4-3) Relationship Between Angle and Dimension

The second optical axis (A2) of the ultraviolet ray reflected by the first reflection unit (71) is shifted from the first optical axis (A1) of the ultraviolet ray of the emission unit (60) by a first angle θ1 toward the one end side in the second direction. It is thus possible to prevent the ultraviolet ray reflected from the first reflection unit (71) from hitting the emission unit (60). As described above, the first angle θ1 is set such that the second optical axis (A2) of the first reflection unit (71) does not overlap with the emission unit (60).

The first angle θ1 is a predetermined angle greater than 0°. By setting the first angle θ1 to be greater than 0°, the ultraviolet ray reflected from the first reflection unit (71) is less likely to hit the emission unit (60) as compared to a case where the first angle θ1 is 0°, and therefore, it is possible to reduce deterioration of the emission unit (60). The first angle θ1 is preferably 1° or more, and may be, for example, 2° or 3°.

A distance in the second direction from the first surface (54) to the starting point P1 of the ultraviolet light from the emission unit (60) is represented by L. A distance in the second direction from the point P1 to the starting point P2 of the light reflected by the first reflection unit (71) is represented by b. In this case, the ultraviolet emission unit (50) satisfies the relationship of Expression (2) below.

$$b \leq L / 2 \qquad ( 2 )$$

If b is greater than L/2, the ultraviolet ray reflected by the first reflection unit (71) may reach the first surface (54). On the other hand, in this embodiment, since b is L/2 or less, it is possible to reduce the likelihood that the ultraviolet ray reflected by the first reflection unit (71) reaches the first surface (54) and make this ultraviolet ray reach the third surface (56). As a result, in the sterilization space (S), the ultraviolet rays reflected by the first reflection unit (71) are emitted to both ends in the first direction, making it possible to utilize the sterilization space (S) effectively.

Further, a distance in the first direction from the starting point P1 of the ultraviolet light from the emission unit (60) to the starting point P2 of the light reflected by the first reflection unit (71) is represented by a. In this case, the ultraviolet emission unit (50) satisfies the relationship of Expression (3) below.

$$First Angle θ 1 < 2 {tan}^{- 1} ( L / 2 a ) \qquad ( 3 )$$

If the first angle θ1 is 2 tan⁻¹ (L/2a) or more, the ultraviolet ray reflected by the first reflection unit (71) may reach the first surface (54). On the other hand, in this embodiment, since the first angle θ1 is less than 2 tan⁻¹ (L/2a), it is possible to reduce the likelihood that the ultraviolet ray reflected by the first reflection unit (71) reaches the first surface (54) and make this ultraviolet ray reach the third surface (56). As a result, in the sterilization space (S), the ultraviolet rays reflected by the first reflection unit (71) are emitted to both ends in the first direction, making it possible to utilize the sterilization space (S) effectively.

The first angle θ1 is preferably 30° or less. This can reliably reduce the likelihood that the ultraviolet ray reflected by the first reflection unit (71) reaches the first surface (54). The first angle θ1 may be 3° or less.

### =(5) Operation of Ultraviolet Emission Unit

The ultraviolet emission unit (50) operates during operation of the air conditioner (10). The control device of the circuit board (63) turns on the LED (61) in the cooling operation and the heating operation. When the indoor fan (33) is operated in the cooling operation or the heating operation, part of the air sucked from the indoor space (I) into the inlet port (41) is sucked into the casing (51) of the ultraviolet emission unit (50). Specifically, air in the air passage (43) flows into the sterilization space (S) through the inflow port (52) of the casing (51).

When the LED (61) is turned on, the ultraviolet ray emitted from the LED (61) is distributed by the reflector (62). The distributed ultraviolet ray is directed to the fourth surface (57) side as light parallel to the first optical axis (A1). The ultraviolet ray reflected by the first reflection unit (71) is directed to the third surface (56) as light parallel to the second optical axis (A2) forming the first angle θ1 with the first optical axis (A1). In this manner, in the sterilization space (S), the ultraviolet ray in the first optical axis (A1) emitted from the emission unit (60) and the ultraviolet ray in the second optical axis (A2) reflected by the first reflection unit (71) are emitted to both the third surface (56) and the fourth surface (57) of the sterilization space (S). The ultraviolet light in the first optical axis (A1) and the second optical axis (A2) propagates in a direction along the longitudinal direction of the sterilization space (S). In addition, the first optical axis (A1) and the second optical axis (A2) forms an angle from the second surface (55) side toward the first surface (54) side. It is thus possible to expand an area irradiated with the ultraviolet ray in the sterilization space (S).

In the sterilization space (S), air flowing in its thickness direction (third direction) is irradiated with the ultraviolet ray. As a result, bacteria and viruses in the air are inactivated. The air in the sterilization space (S) flows out through the outflow port (53). The air that has flowed out is cooled or heated by the indoor heat exchanger (32) and is then supplied to the indoor space (I) through the outlet port (42).

### (6) Advantages of Embodiment

The second optical axis (A2) of the light reflected by the first reflection unit (71) is shifted from the first optical axis (A1) of the ultraviolet ray of the emission unit (60) by the first angle θ1 toward one end side in the second direction orthogonal to the first direction. It is thus possible to prevent the light reflected by the first reflection unit (71) from hitting the emission unit (60). As a result, it is possible to prevent deterioration of the emission unit (60) due to the ultraviolet ray. In addition, since the ultraviolet ray reflected by the first reflection unit (71) returns to one end side in the first direction, it is possible to reduce the likelihood that the ultraviolet ray leaks from the first surface (54) side to the outside.

The ultraviolet emission unit (50) includes only one reflection unit that reflects the ultraviolet ray emitted from the emission unit (60). Thus, as compared to a configuration in which ultraviolet rays are emitted from a large number of reflection units, attenuation of the ultraviolet rays due to reflection can be reduced.

A first length of the sterilization space (S) in the first direction is greater than a second length of the sterilization space (S) in the second direction. This increases a travel distance of the ultraviolet ray emitted from the emission unit (60) to the first reflection unit (71). This ultraviolet ray is an ultraviolet ray before attenuation due to reflection by the first reflection unit (71). Thus, the ultraviolet ray having a high illuminance can be emitted over the entire sterilization space (S) in the longitudinal direction. As a result, the sterilization effect on air in the sterilization space (S) can be enhanced.

A third length in the third direction orthogonal to the first direction and the second direction of the sterilization space (S) is less than the first length and the second length. The casing (51) is configured such that air flows through the sterilization space (S) from one side to the other side in the third direction. Specifically, the air flows through the sterilization space (S) along the third direction. Thus, a flow path length of the air passing through the sterilization space (S) is short, and a flow path cross-sectional area is large. It is thus possible to reduce the flow resistance of the air. As a result, it is possible to reduce the likelihood that the power required to drive the indoor fan (33) increases due to pressure loss.

A relationship b≤L/2 is satisfied, where L is the distance in the second direction from the first surface (54), which is the inner surface on one end side in the second direction of the sterilization space (S), to the starting point P1 of the ultraviolet light of the emission unit (60), and b is the distance in the second direction from the starting point P1 to the starting point P2 of the light reflected by the first reflection unit (71). In this manner, setting the distance b to be short can prevent the ultraviolet ray reflected by the first reflection unit (71) from hitting the first surface (54). As a result, the reflected light area in the sterilization space (S) can be expanded. In addition, it is possible to reduce the likelihood that the reflected light leaks from the first surface (54) side to the outside.

A relationship First Angle θ1<2 tan⁻¹ (L/2a) is satisfied, where L is the distance in the second direction from the first surface (54), which is the inner surface on one end side in the second direction of the sterilization space (S), to the starting point P1 of the ultraviolet light of the emission unit (60), and a is the distance in the first direction from the starting point P1 to the starting point P2 of the light reflected by the first reflection unit (71). In this manner, setting the first angle θ1 to be small can prevent the ultraviolet ray reflected by the first reflection unit (71) from hitting the first surface (54). As a result, the reflected light area in the sterilization space (S) can be expanded. In addition, it is possible to reduce the likelihood that the reflected light leaks from the first surface (54) side to the outside.

The first reflection unit (71) has an ultraviolet ray reflectance of 50% or more. This can reduce the attenuation of the illuminance of the ultraviolet ray due to reflection of the ultraviolet ray by the first reflection unit (71).

The ultraviolet emission unit (50) is provided in the air conditioner (10). Thus, the target air of the air conditioner (10) can be sterilized by the ultraviolet emission unit (50).

The air conditioner (10) includes the indoor casing (30a) which is the air conditioning casing. The casing (51) of the ultraviolet emission unit (50) is disposed inside the indoor casing (30a). Thus, the sterilization space (S) is located inside both the casing (51) of the ultraviolet emission unit (50) and the air conditioning casing (30a). Accordingly, it is possible to reduce the likelihood that the ultraviolet ray in the sterilization space (S) leaks to the outside of the air conditioning casing (30a).

The casing (51) of the ultraviolet emission unit (50) extends along the longitudinal direction of the air conditioning casing (30a). This can ensure a sufficient space for disposing the ultraviolet emission unit (50) inside the air conditioning casing (30a). The length of the sterilization space (S) in the longitudinal direction can be extended, so that the air sterilization efficiency can be enhanced.

The ultraviolet emission unit (50) is disposed upstream of the indoor heat exchanger (32). Thus, the ultraviolet emission unit (50) is less likely to be affected by heat of the indoor heat exchanger (32) during the operation of the air conditioner (10).

### (7) Variations

The above-described embodiment may be modified as follows. Differences from the above-described embodiment will be described below.

### (7-1) Variation 1

The emission unit (60) of the above-described embodiment may be configured as follows.

### (7-1-1) Variation 1A

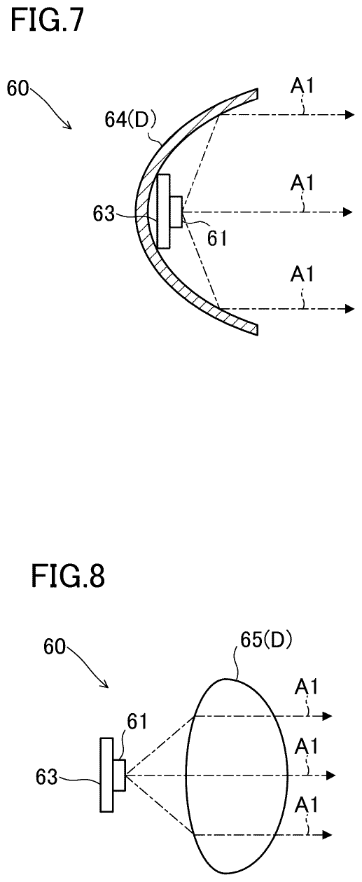

In the emission unit (60) of Variation 1A illustrated in FIG. 7, the LED (61) faces the first reflection unit (71) side. The circuit board (63) is located closer to the third surface (56) side than the LED (61). The curved inner surface of the reflector (64) faces the first reflection unit (71) side. The ultraviolet ray emitted from the LED (61) to the first reflection unit (71) side is reflected by the inner surface of the reflector (64), and is then sent to the first reflection unit (71) side along the first optical axis (A1).

### (7-1-2) Variation 1B

The emission unit (60) of Variation 1B illustrated in FIG. 8 includes a condensing lens (65) which is the light distribution control unit (D). The condensing lens (65) condenses the ultraviolet ray emitted from the LED (61) and distributes the ultraviolet ray to the first reflection unit (71) side. The ultraviolet ray condensed by the condensing lens (65) is sent to the first reflection unit (71) side along the first optical axis (A1). The condensing lens (65) may be a Fresnel lens having a serrated cross-sectional shape passing through its axial center. The condensing lens (65) may be a total internal reflection (TIR) lens. The TIR lens has a surface which totally reflects the ultraviolet ray in addition to a surface which condenses the ultraviolet ray emitted from the LED (61).

### (7-1-3) Variation 1C

The light distribution control unit (D) may have the reflector (62) and the condensing lens (65), described above, and may be configured to distribute the ultraviolet ray to the first reflection unit (71) side by these components.

### (7-2) Variation 2

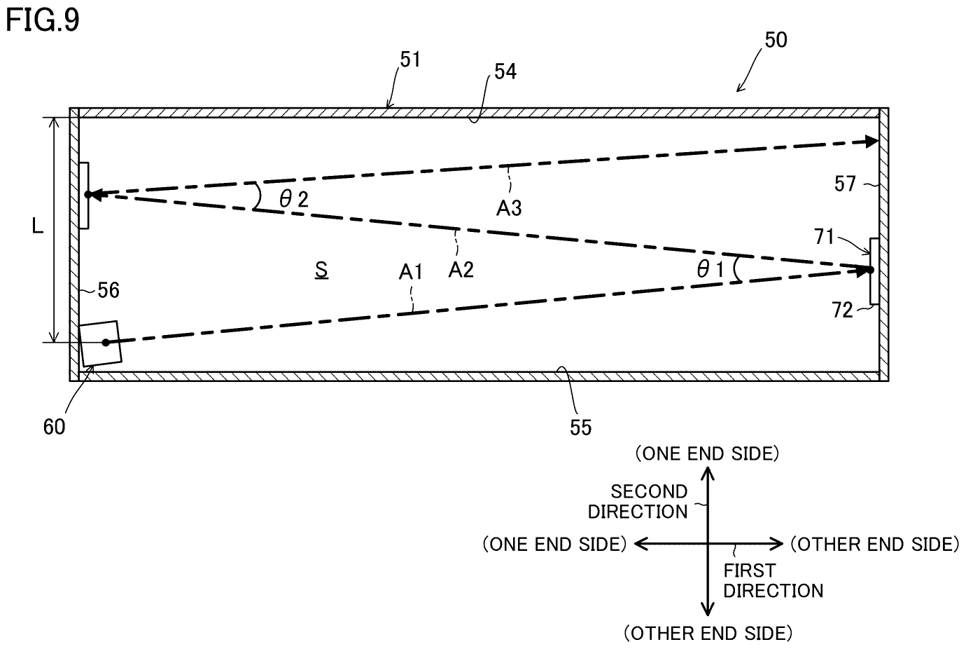

The ultraviolet emission unit (50) of Variation 2 illustrated in FIG. 9 includes a second reflection unit (80). The second reflection unit (80) is disposed on one end side in the first direction of the sterilization space (S) and reflects the ultraviolet ray emitted from the first reflection unit (71) toward the other end side.

The second reflection unit (80) is disposed between an intermediate position in the first direction of the sterilization space (S) and the third surface (56). The second reflection unit (80) is disposed in the vicinity of the third surface (56). The second reflection unit (80) is disposed between an intermediate position in the second direction of the sterilization space (S) and the first surface (54). The second reflection unit (80) is preferably fixed to the third surface (56).

A third optical axis (A3) of the light reflected by the second reflection unit (80) is shifted from the second optical axis (A2) of the first reflection unit (71) by a second angle θ2 toward one end side in the second direction. Thus, in the sterilization space (S), the area of the ultraviolet ray emitted from the emission unit (60) expands toward the one end side in the second direction. As a result, the area where the ultraviolet ray is emitted can expand in the second direction, and the sterilization efficiency can be enhanced.

The second angle θ2 is set such that the ultraviolet ray reflected by the second reflection unit (80) returns to the fourth surface (57). In other words, the third optical axis (A3) of the second reflection unit (80) is directed to the fourth surface (57). As a result, the reflected light area in the sterilization space (S) can be expanded. In addition, it is possible to reduce the likelihood that the reflected light leaks from the first surface (54) side to the outside.

The third optical axis (A3) of the light reflected by the second reflection unit (80) is directed to a corner portion between the first surface (54) and the fourth surface (57). Thus, the sterilization efficiency can be enhanced in an area around the corner portion.

The second angle θ2 merely needs to be greater than 0°. The second angle θ2 is preferably 1° or more, and may be, for example, 2° or 3°. That is, the second angle θ2 may be 3° or less. The second angle θ2 may be the same as or different from the first angle θ1.

### (7-3) Variation 3

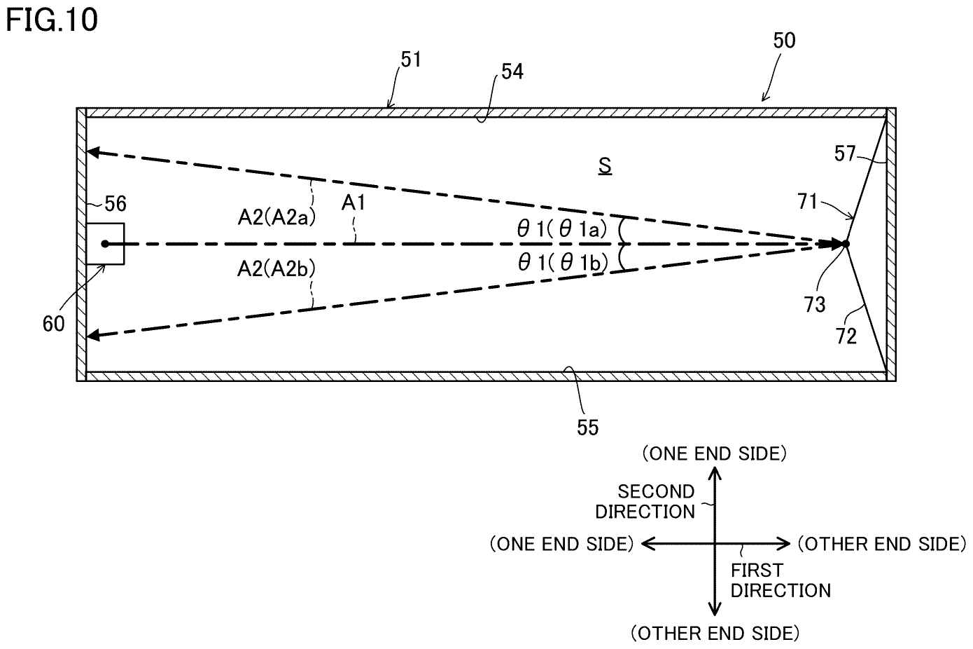

In Variation 3 illustrated in FIG. 10, the emission unit (60) is provided at the intermediate position in the second direction. The first optical axis (A1) of the emission unit (60) extends along the first direction.

The first reflection unit (71) has a bent portion (73) at an intermediate portion in the second direction. The bent portion (73) is located at a top portion of the reflection surface (72) protruding toward the third surface (56) side. The bent portion (73) is located at a position overlapping with the emission unit (60) in the first direction. The reflection surface (72) of the first reflection unit (71) reflects the ultraviolet ray in two directions, with the bent portion (73) serving as a boundary. Specifically, the second optical axis (A2) of the light reflected by the first reflection unit (71) includes an optical axis A2 (A2a) and an optical axis A2 (A2b). The optical axis A2 (A2a) is shifted from the first optical axis (A1) by an angle θ1a toward the first surface (54) side. The optical axis A2 (A2b) is shifted from the first optical axis (A1) by an angle θ1b toward the second surface (55) side.

In this configuration as well, it is possible to reduce the likelihood that the light reflected by the first reflection unit (71) hits the emission unit (60), thereby reducing deterioration of the emission unit (60).

Since the optical axis A2 (A2a) and the optical axis A2 (A2b) extend to both ends in the second direction with respect to the emission unit (60), the area irradiated with the ultraviolet ray in the sterilization space (S) can be expanded.

The optical axis A2 (A2a) is directed to a corner portion between the first surface (54) and the third surface (56). Thus, the sterilization efficiency can be enhanced in an area around the corner portion. The optical axis A2 (A2b) is directed to a corner portion between the third surface (56) and the second surface (55). Thus, the sterilization efficiency can be enhanced in an area around the corner portion.

The angle θ1a and the angle θ1b merely need to be greater than 0°. The angle θ1a and the angle θ1b are preferably 1° or more, and may be, for example, 2° or 3°. That is, the angle θ1a and the angle θ1b may be 3° or less.

### (7-4) Variation 4

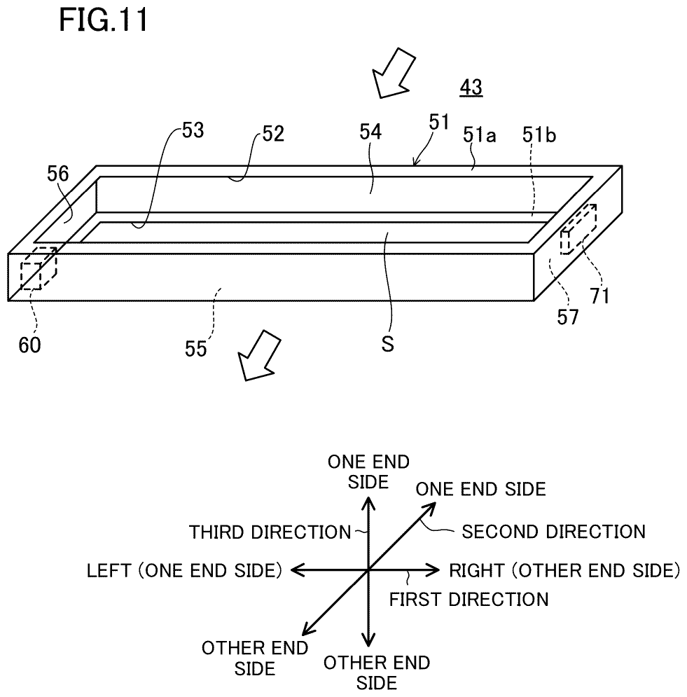

The casing (51) of Variation 4 illustrated in FIG. 11 is configured such that air flows through the sterilization space (S) from one side to the other side in the third direction, similarly to the above-described embodiment. On the other hand, in Variation 4, the direction of air flowing through the sterilization space (S) is inclined with respect to the third direction. The angle formed between the air flow direction and the third direction may be any angle as long as it is 45° or less. In this configuration as well, a flow path length of the air passing through the sterilization space (S) is short, and a flow path cross-sectional area is large. It is thus possible to reduce the flow resistance of the air. As a result, it is possible to reduce the likelihood that the power required to drive the indoor fan (33) increases due to pressure loss.

### (7-5) Variation 5

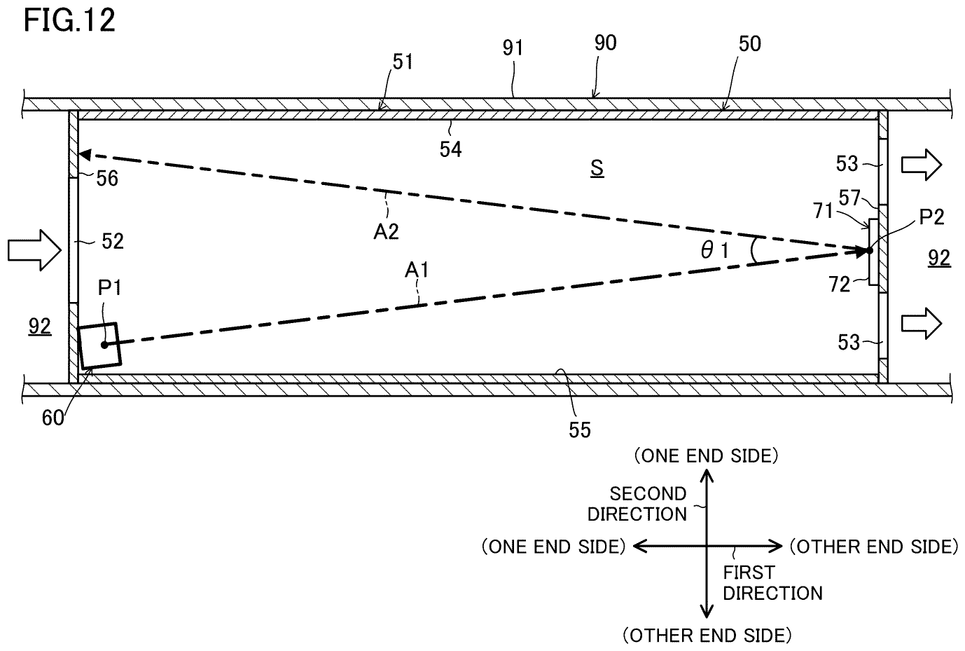

In Variation 5 illustrated in FIG. 12, the ultraviolet emission unit (50) is applied to an air duct (90). The air duct (90) includes a duct body (91) and the ultraviolet emission unit (50). A duct flow path (92) through which air flows is formed inside the duct body (91). The duct body (91) is formed in a tubular shape extending along an air flow direction. The duct body (91) may have a cylindrical shape or a rectangular tubular shape. The duct body (91) may be made of a hard material such as resin or iron, or may be made of a flexible material such as a hose.

The casing (51) of the ultraviolet emission unit (50) is disposed in the duct flow path (92) of the duct body (91). The casing (51) extends in the air flow direction in the duct flow path (92). Specifically, the first direction, which is the longitudinal direction of the casing (51), corresponds to the air flow direction in the duct flow path (92) or a tube axis direction. The second direction, which is the lateral direction of the casing (51), corresponds to a direction orthogonal to the air flow in the duct flow path (92). The casing (51) of this example is formed in a tubular shape along the inner peripheral surface of the duct body (91). This configuration maximizes the volume of the sterilization space (S) of the casing (51).

The inflow port (52) is formed in the third surface (56) of the casing (51). The inflow port (52) opens to the upstream side of the duct flow path (92). The inflow port (52) is preferably located at a position not overlapping with the emission unit (60) in the first direction. The number of inflow ports (52) may be one or may be two or more.

The outflow port (53) is formed in the fourth surface (57) of the casing (51). The outflow port (53) opens to the downstream side of the duct flow path (92). The outflow port (53) is preferably located at a position not overlapping with the emission unit (60) in the first direction. The number of outflow ports (53) may be one or may be two or more.

The casing (51) is configured such that air flows through the sterilization space (S) from one side to the other side in the first direction. Specifically, the casing (51) is configured such that air flows through the sterilization space (S) along the first direction. The air in the sterilization space (S) flows in the same direction as that of the air in the duct flow path (92). Thus, the air in the duct flow path (92) passes through the casing (51) without changing its direction. This can reduce the flow resistance in the casing (51).

In this example as well, the second optical axis (A2) of the light reflected by the first reflection unit (71) is shifted from the first optical axis (A1) of the ultraviolet ray of the emission unit (60) by the first angle θ1. It is thus possible to reduce the likelihood that the ultraviolet ray hits the emission unit (60), thereby reducing deterioration of the emission unit (60). Similarly to Variation 4, the air flowing through the casing (51) may be inclined with respect to the first direction. The angle formed between the air flow direction and the first direction may be any angle as long as it is 45° or less.

### (7-6) Variation 6

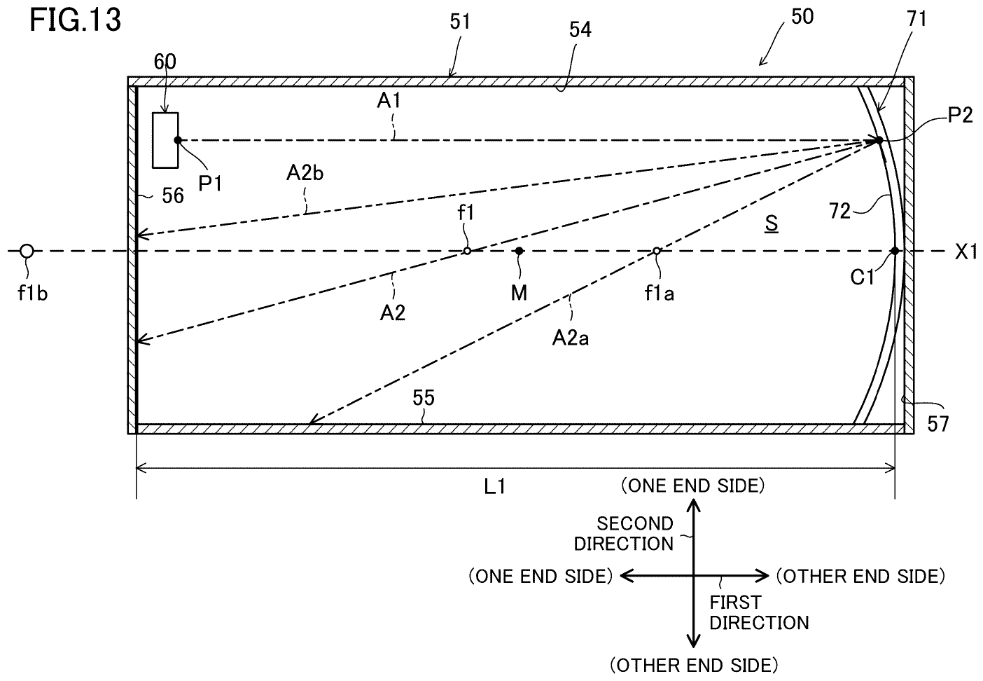

Variation 6 illustrated in FIG. 13 differs from the above-described embodiment in the configuration of the first reflection unit (71). In the sterilization space (S) of Variation 6, the emission unit (60) is provided on the third surface (56) side, and the first reflection unit (71) is provided on the fourth surface (57) side.

The emission unit (60) is disposed so as to be shifted in the second direction from an optical axis X1. Specifically, the emission unit (60) of this example is disposed near one end side (first surface (54)) in the second direction. The emission unit (60) emits the ultraviolet ray in the first direction. The emission unit (60) may emit the ultraviolet ray such that the ultraviolet ray is shifted inward in the second direction from the first direction by a predetermined angle.

The first reflection unit (71) extends across both ends in the second direction of the sterilization space (S). The first reflection unit (71) has a first reflection surface (72). The first reflection surface (72) is formed in an arc shape recessed toward the other end side in the first direction when viewed in the cross section in the third direction orthogonal to the first direction and the second direction. That is, the first reflection surface (72) has a curved shape recessed toward the other end side in the first direction when viewed in the cross section in the third direction. The first reflection surface (72) preferably has a curved shape recessed toward the other end side in the first direction when viewed in the cross section in the second direction. In other words, the first reflection surface (72) preferably has a spherical shape or a parabolic shape. This can reduce the likelihood that the ultraviolet ray reflected by the first reflection surface (72) leaks from the sterilization space (S) to the outside in the third direction.

The first reflection surface (72) has a first curvature radius R1. In FIG. 13, C1 is the midpoint of the arc surface of the first reflection surface (72), and X1 is the optical axis of the first reflection surface (72) itself.

Similarly to the above-described embodiment, the second optical axis (A2) of the ultraviolet ray reflected by the first reflection unit (71) is shifted from the first optical axis (A1) of the ultraviolet ray of the emission unit (60) by a first angle θ1 toward the other end side in the second direction. It is thus possible to prevent the ultraviolet ray reflected from the first reflection unit (71) from hitting the emission unit (60). Preferably, the second optical axis (A2) is shifted inwardly in the second direction from the first optical axis (A1) by a predetermined angle.

A distance from C1 to the third surface (56) is represented by L1. In this case, it is preferable that the first reflection unit (71) is configured to satisfy a relationship R1≥L1. If R1 is less than L1, the focal point f1 of the first reflection surface (72) is closer to the first reflection unit (71) than the midpoint M on X1, as shown by, for example, a focal point f1a in FIG. 13. As a result, the second optical axis (A2) of the ultraviolet ray reflected by the first reflection unit (71) deviates in the second direction in the sterilization space (S), as shown by the optical axis (A2a) in FIG. 13. On the other hand, by setting R1≥L1, it is possible to reduce the likelihood that the ultraviolet ray reflected from the first reflection unit (71) deviates in the second direction in the sterilization space (S).

The first reflection unit (71) is preferably configured to satisfy a relationship R1≤2×L1. If R1 is greater than 2×L1, the focal point f1 of the first reflection surface (72) is located to the left (one end side in the first direction) of the third surface (56), as shown by, for example, a focal point f1b in FIG. 13. As a result, the ultraviolet ray reflected by the first reflection unit (71) (second optical axis (A2)) approaches the emission unit (60), as shown by the optical axis (A2b) in FIG. 13. On the other hand, by setting R1≤2× L, it is possible to reduce the likelihood that the ultraviolet ray reflected from the first reflection unit (71) approaches the emission unit (60), thereby reducing deterioration of the emission unit (60).

### (7-7) Variation 7

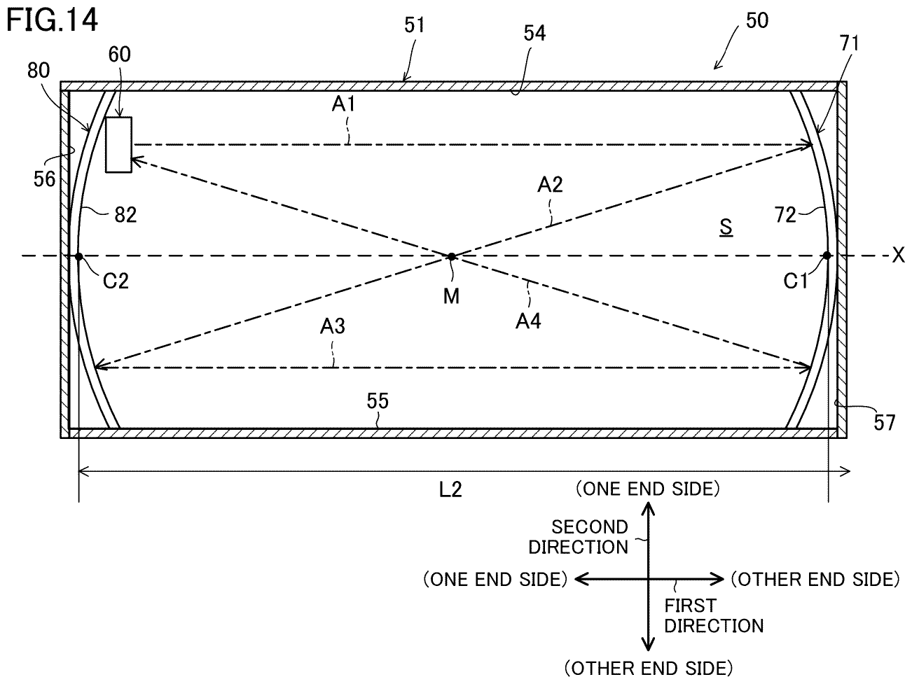

Variation 7 further has the second reflection unit (80) in the configuration of Variation 6. As illustrated in FIG. 14, the second reflection unit (80) is provided on the third surface (56) side. The second reflection unit (80) extends across both ends in the second direction of the sterilization space (S). The second reflection unit (80) has a second reflection surface (82). The second reflection surface (82) is formed in an arc shape recessed toward one end side in the first direction when viewed in the cross section in the third direction. That is, the second reflection surface (82) has a curved shape recessed toward one end side in the first direction when viewed in the cross section in the third direction. The second reflection surface (82) preferably has a curved shape recessed toward one end side in the first direction when viewed in the cross section in the second direction. In other words, the second reflection surface (82) preferably has a spherical shape or a parabolic shape. This can reduce the likelihood that the ultraviolet ray reflected by the second reflection surface (82) leaks from the sterilization space (S) to the outside in the third direction.

The second reflection surface (82) is opposed to the first reflection surface (72) in the first direction. The second reflection surface (82) has a second curvature radius R2.

In FIG. 14, C1 is the midpoint of the arc surface of the first reflection surface (72); C2 is the midpoint of the arc surface of the second reflection surface (82); and X is the optical axis of the first reflection surface (72) and the second reflection surface (82) themselves.

A distance from C1 to C2 is represented by L2. In this case, it is preferable that the first reflection unit (71) is configured to satisfy a relationship R1≥L2, and more preferably to satisfy a relationship R1≤2×L2. The reason is the same as that described in the above Variation 6.

It is preferable that the second reflection unit (80) satisfies a relationship R2≥L2. This is because if R2 is less than L2, the focal point of the second reflection surface (82) approaches the second reflection unit (80), and the optical axis of the second reflection unit (80) deviates from one end side in the second direction in the sterilization space (S), similarly to the first reflection unit (71) described above. On the other hand, by setting R2≥L2, it is possible to reduce the likelihood that the ultraviolet ray reflected from the second reflection unit (80) deviates in the second direction in the sterilization space (S).

The first reflection unit (71) and the second reflection unit (80) are configured such that the reflected ultraviolet rays are within the sterilization space (S). Here, in the first reflection unit (71) and the second reflection unit (80), the total number of times of reflection is preferably three or more, and more preferably seven or more.

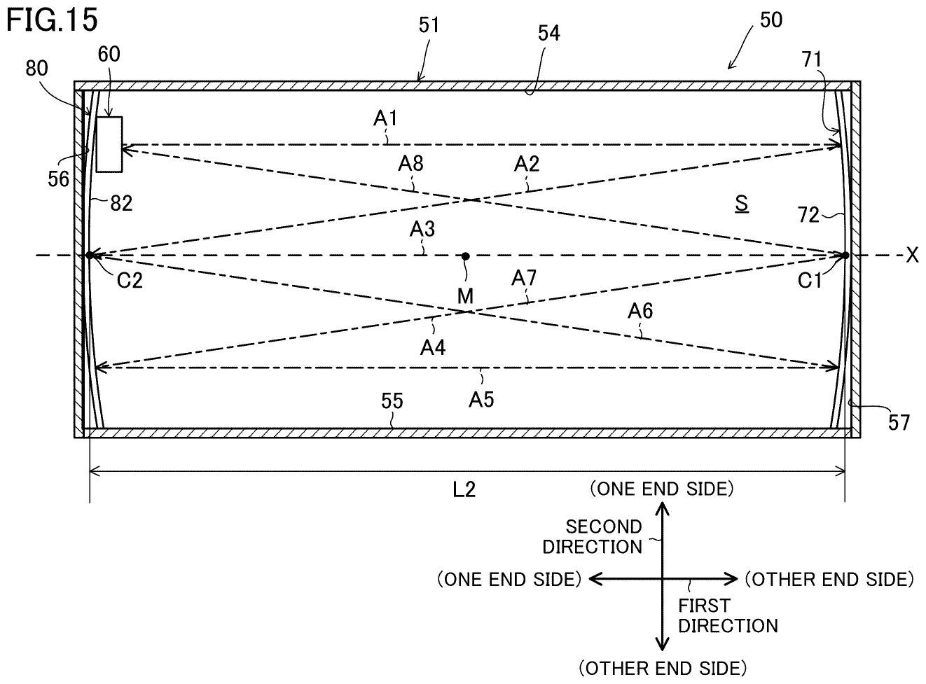

When R1 and R2 are equal to each other (R=R1=R2), it is preferable that the first reflection unit (71) and the second reflection unit (80) satisfy a relationship L2<R, and more preferably to satisfy a relationship R<2×L2. This is because if L2=R, as illustrated in FIG. 14, the optical axis (optical axis (A4) in FIG. 14) of the ultraviolet ray reflected a second time by the first reflection unit (71) is more likely to reach the emission unit (60). Further, if R=2×L2, as illustrated in FIG. 15, the optical axis (optical axis (A8) in FIG. 14) of the ultraviolet ray reflected a third time by the first reflection unit (71) is more likely to reach the emission unit (60). On the other hand, by satisfying the relationship L2<R<2×L2, it is possible to reduce the likelihood that the ultraviolet ray reflected from the first reflection unit (71) reaches the emission unit (60).

If R1 and R2 differ from each other, it is possible to prevent the ultraviolet ray reflected from the first reflection unit (71) from reaching the emission unit (60), as illustrated in FIG. 14 or 15. Thus, the first reflection unit (71) and the second reflection unit (80) may be configured such that the curvature radii R1 and R2 differ from each other.

### (8) Other Embodiments

The flow path forming member of the ultraviolet emission unit (50) may be the air conditioning casing (30a) of the air conditioner (10), or may be formed by part of the air conditioning casing (30a) and other components of the air conditioner (10). For example, the first surface (54) and second surface (55) of the flow path forming member illustrated in FIG. 5 may be formed by the above components.

The flow path forming member of the ultraviolet emission unit (50) may be only part of the air conditioning casing (30a) of the air conditioner (10). The flow path forming member of the ultraviolet emission unit (50) does not necessarily have to be frame-shaped, as long as it has a surface that defines the sterilization space (S) through which air passes.

The flow path forming member may be formed by part of the duct body (91) of the air duct (90). In this case, the sterilization space (S) is formed inside the air conditioning casing (30a) or inside the duct body (91).

The first length of the sterilization space (S) in the first direction may be shorter than the second length of the sterilization space (S) in the second direction.

The air conditioner (10) is a pair-type air conditioner including one indoor unit (30) and one outdoor unit (20). However, the air conditioner (10) may be of an indoor multi-type having two or more indoor units (30) or an outdoor multi-type having two or more outdoor units (20).

The air conditioner (10) may be a ventilator that provides air ventilation, an air purifier that purifies air, or a humidity control apparatus that humidifies or dehumidifies air. In other words, the “air conditioning” described herein means not only temperature control of air, but also ventilation of air, purification of air, and humidity control of air.

While the embodiments and variations thereof have been described above, it will be understood that various changes in form and details may be made without departing from the spirit and scope of the claims. The elements according to the embodiment, variations thereof, and the other embodiments may be combined and replaced with each other.

The expressions such as “first,” “second,” “third,” . . . , described above are used to distinguish the terms to which these expressions are given, and do not limit the number and order of the terms.

### INDUSTRIAL APPLICABILITY

As described above, the present disclosure is useful for an ultraviolet emission unit, an air conditioner, and an air duct.

### EXPLANATION OF REFERENCES

10 Air Conditioner
30a Indoor Casing (Air Conditioning Casing)
43 Air Passage
50 Ultraviolet Emission Unit
51 Casing (Flow Path Forming Member)
54 First Surface
60 Emission Unit
71 First Reflection Unit
80 Second Reflection Unit
90 Air Duct
S Sterilization Space

1. An ultraviolet emission unit comprising:
a flow path forming member forming a sterilization space through which air flows;
an emission unit disposed on one end side in a first direction of the sterilization space and configured to emit an ultraviolet ray toward the other end side in the first direction; and
a first reflection unit disposed on the other end side in the first direction of the sterilization space and configured to reflect the ultraviolet ray emitted from the emission unit such that the ultraviolet ray returns to the one end side in the first direction,
an optical axis of light reflected by the first reflection unit being shifted from an optical axis of the ultraviolet ray of the emission unit by a first angle θ1 toward one end side in a second direction orthogonal to the first direction.

2. The ultraviolet emission unit of claim 1, wherein
a first length of the sterilization space in the first direction is greater than a second length of the sterilization space in the second direction.

3. The ultraviolet emission unit of claim 2, wherein
a third length in a third direction orthogonal to the first direction and the second direction of the sterilization space is less than the first length and the second length, and
the flow path forming member is configured such that air flows from one side to the other side in the third direction.

4. The ultraviolet emission unit of claim 1, wherein
the flow path forming member is configured such that air flows through the sterilization space from one side to the other side in the first direction.

5. The ultraviolet emission unit of claim 1, wherein
a relationship b≤L/2 is satisfied,
where L is a distance in the second direction from a first surface, which is an inner surface on the one end side in the second direction of the sterilization space, to a starting point P1 of ultraviolet light of the emission unit, and b is a distance in the second direction from the starting point P1 to a starting point P2 of the light reflected by the first reflection unit.

6. The ultraviolet emission unit of claim 1, wherein
a relationship, the first angle θ1<2 tan⁻¹ (L/2a), is satisfied,
where L is a distance in the second direction from a first surface, which is an inner surface on the one end side in the second direction of the sterilization space, to a starting point P1 of ultraviolet light of the emission unit, and a is a distance in the first direction from the starting point P1 to a starting point P2 of the light reflected by the first reflection unit.

7. The ultraviolet emission unit of claim 1, further comprising:
a second reflection unit disposed on one end side in the first direction of the sterilization space and configured to reflect the ultraviolet ray, emitted from the first reflection unit, toward the other end side in the first direction, wherein
an optical axis of light reflected by the second reflection unit is shifted from the optical axis of the first reflection unit by a second angle θ2 toward the one end side in the second direction.

8. The ultraviolet emission unit of claim 1, wherein
the first angle θ1 is 30 degrees or less.

9. The ultraviolet emission unit of claim 1, wherein
the first reflection unit has an ultraviolet ray reflectance of 50% or more.

10. An air conditioner comprising the ultraviolet emission unit of claim 1.

11. The air conditioner of claim 10, further comprising:
an air conditioning casing in which an air passage is formed, wherein
the flow path forming member is disposed in the air passage.

12. An air duct comprising the ultraviolet emission unit of claim 1.

13. The ultraviolet emission unit of claim 2, wherein
the flow path forming member is configured such that air flows through the sterilization space from one side to the other side in the first direction.

14. The ultraviolet emission unit of claim 2, wherein
a relationship b≤L/2 is satisfied,
where L is a distance in the second direction from a first surface, which is an inner surface on the one end side in the second direction of the sterilization space, to a starting point P1 of ultraviolet light of the emission unit, and b is a distance in the second direction from the starting point P1 to a starting point P2 of the light reflected by the first reflection unit.

15. The ultraviolet emission unit of claim 3, wherein
a relationship b≤L/2 is satisfied,
where L is a distance in the second direction from a first surface, which is an inner surface on the one end side in the second direction of the sterilization space, to a starting point P1 of ultraviolet light of the emission unit, and b is a distance in the second direction from the starting point P1 to a starting point P2 of the light reflected by the first reflection unit.

16. The ultraviolet emission unit of claim 4, wherein
a relationship b≤L/2 is satisfied,
where L is a distance in the second direction from a first surface, which is an inner surface on the one end side in the second direction of the sterilization space, to a starting point P1 of ultraviolet light of the emission unit, and b is a distance in the second direction from the starting point P1 to a starting point P2 of the light reflected by the first reflection unit.

17. The ultraviolet emission unit of claim 2, wherein
a relationship, the first angle θ1<2 tan⁻¹ (L/2a), is satisfied,
where L is a distance in the second direction from a first surface, which is an inner surface on the one end side in the second direction of the sterilization space, to a starting point P1 of ultraviolet light of the emission unit, and a is a distance in the first direction from the starting point P1 to a starting point P2 of the light reflected by the first reflection unit.

18. The ultraviolet emission unit of claim 3, wherein
a relationship, the first angle θ1<2 tan⁻¹ (L/2a), is satisfied,
where L is a distance in the second direction from a first surface, which is an inner surface on the one end side in the second direction of the sterilization space, to a starting point P1 of ultraviolet light of the emission unit, and a is a distance in the first direction from the starting point P1 to a starting point P2 of the light reflected by the first reflection unit.

19. The ultraviolet emission unit of claim 4, wherein
a relationship, the first angle θ1<2 tan⁻¹ (L/2a), is satisfied,
where L is a distance in the second direction from a first surface, which is an inner surface on the one end side in the second direction of the sterilization space, to a starting point P1 of ultraviolet light of the emission unit, and a is a distance in the first direction from the starting point P1 to a starting point P2 of the light reflected by the first reflection unit.

20. The ultraviolet emission unit of claim 5, wherein
a relationship, the first angle θ1<2 tan⁻¹ (L/2a), is satisfied,
where L is a distance in the second direction from a first surface, which is an inner surface on the one end side in the second direction of the sterilization space, to a starting point P1 of ultraviolet light of the emission unit, and a is a distance in the first direction from the starting point P1 to a starting point P2 of the light reflected by the first reflection unit.
